(19) United States
(12) Patent Application Publication
(10) Pub. No.: US 2023/0054687 A1
(43) Pub. Date: Feb. 23, 2023

(54) Remote Detection Apparatus

(71) Applicants: Azardokht Mazaheri, Tehran (IR); Mehdi Hakkak, Tehran (IR)

(72) Inventors: Azardokht Mazaheri, Tehran (IR); Mehdi Hakkak, Tehran (IR)

(73) Assignee: Petro Farzan Apadana company, Tehran (IR)

(21) Appl. No.: 17/958,897

(22) Filed: Oct. 3, 2022

Related U.S. Application Data
(63) Continuation-in-part of application No. PCT/IB2020/061573, filed on Dec. 7, 2020.

(51) Int. Cl. G01N 33/18; G01N 21/39

(52) U.S. Cl. CPC G01N 33/1833; G01N 21/39; G01N 2021/399; G01N 2021/1793

(57) ABSTRACT

A remote detection apparatus to detect an oil pollutant comprises a light emitting module, an optical receiving device including a first lens, a band-pass filter, at least two second lenses and at least an optical fiber configured to receiving a reflected beam, a detector module, and a micro-controller. The apparatus further comprises a house including an optical window configured to protect the light emitting module, the optical receiving device, the detector module, and the micro-controller from harsh environmental conditions.

The present disclosure is a continuation-in-part of PCT/IB2020/061573 filed Dec. 7, 2020, entitled “Remote Detection Apparatus” that claims priority from IR Patent Application, Application No 139950140003000229, filed on 5 Apr. 2020, which is incorporated by reference herein in its entirety.

### TECHNICAL FIELD

The present disclosure is related to a remote detection apparatus to detect a water pollutant, specially an oil spill on water.

### BACKGROUND ART

More than two million tons of petroleum are produced annually in the world. The petroleum contains many different kinds of petrochemical hydrocarbons which are one of the most prevalent organic groups. The petrochemical hydrocarbons are one of the most common groups of organic pollutants in the environment due to their solubility, volatility, and biodegradability and are known to be toxic for many organisms. These hydrocarbons are naturally present in chemicals that used by humans for a variety of activities, including refueling vehicles and heating homes.

These days, soils and groundwater sources around refineries, refueling stations and fuel transfer facility pipelines are contaminated with petrochemical hydrocarbons, which are important environmental issues. Actually, Leakage of these hydrocarbons under the influence of capillary and gravity forces leads to vertical movement in unsaturated soils and fills soil pores so that if the amount of leakage is high, the liquid phase reaches the surface of water and accumulates on the surface of water then the petrochemicals hydrocarbons moves with the ground water and due to its lower specific gravity than the water remains floating on the surface of the water.

Entrance of these substances into soil and groundwater by refineries, runoffs, or leakages from underground fuel tanks, and profoundly threaten the health of humanity as well as the environment. Therefore, there is a need for an apparatus that detect these pollutants in water. Different technologies have been applied to detect oil in water, however among them laser remote sensing technology have an interesting and specified characteristic for on-line and real-time detection of petrochemical hydrocarbons and other water pollutants that have a fluorescent characteristic that causes rapid control of these pollutants in environment, especially in water. Hence, developing a remote and real-time apparatus with an ability to detect the petrochemical hydrocarbons and the other water pollutants on the surface of water is required. This apparatus can rapidly inform about the water pollution and also supply recordable data for later investigations.

### SUMMARY OF INVENTION

This summary is intended to provide an overview of the subject matter of this application, and is not intended to identify essential elements or key elements of the subject matter, nor is it intended to be used to determine the scope of the claimed implementations. The proper scope of this application may be ascertained from the claims set forth below in view of the detailed description below and the drawings.

In one general aspect, the present disclosure is directed to an exemplary remote detection apparatus to detect a water pollutant. The exemplary remote detection apparatus may comprise a light emitting module, the light emitting module may configure to sense a surface of water and induce a fluorescent property of the water pollutant to produce a reflected beam, an optical receiving device, the optical receiving device may configure to receive the reflected beam, a detector module, the detector module may configure to detect the reflected beam and produce a fluorescence spectrum, a programmable device configured to analyze the fluorescence spectrum and set an alarm, the programmable device may include one or more processors, at least a memory, a computing program, and at least one connection port, and a micro-controller, the micro-controller may configure to control a performance of the light emitting module and digitize the fluorescence spectrum.

The above general aspect may have one or more of the following features. In an exemplary implementation, the exemplary laser remote detection apparatus may further comprise a house that may configure to encompass the light emitting module, the optical receiving device, the detector module, and the micro-controller such that the house comprises at least an optical window that the optical window may configure to provide a path for emission an emitted beam to the surface of water and receiving the reflected beam. In an exemplary implementation, the light emitting module and the optical receiving device may have a tilt angle with respect to an optical axis of the optical receiving device. In an exemplary implementation, the light emitting module and the optical receiving device may have a tilt angle in a range of 2-10 degrees with respect to an optical axis of the optical receiving device. In an exemplary implementation, the light emitting module and the optical receiving device may have a 2° tilt angle with respect to an optical axis of the optical receiving device. In an exemplary implementation, the light emitting module may be a pulsed diode laser. In an exemplary implementation, the light emitting module may be a pulsed diode laser that the laser may configure to emit the emitted beam in a range of 350-450 nm. In an exemplary implementation, the optical receiving device may comprise a first lens, at least one filter, at least two second lenses, and at least an optical fiber. In an exemplary implementation, the filter may include a band-pass filter that the filter may configure to pass an oil pollutant reflected beam. In an exemplary implementation, the filter may include a high-pass filter that the filter may configure to eliminate the emitted beam and pass a non-oil pollutant reflected beam. In an exemplary implementation, the two second lenses may configure to arrange the beam along the optical axis of the optical receiving device.

In another general aspect, the present disclosure is directed to an exemplary remote detection apparatus to detect an oil spill. The exemplary remote detection apparatus may comprise a light emitting module, the light emitting module may configure to sense a surface of water and induce a fluorescent property of the oil spill to produce an oil spill reflected beam, an optical receiving device configured to receive the reflected beam such that a tilt angle between the optical receiving device and the light emitting module with respect to an optical axis of the optical receiving device may be in a range of 2 to 10 degrees that the tilt angle may configure to adjust a distance between the apparatus and the oil spill, the optical receiving device may include a plano-convex lens, at least a filter, at least two bi-convex lenses, and at least an optical fiber such that the filter may include a band-pass filter that may configure to pass the oil spill reflected beam, a detector module, the detector module may configure to detect the oil spill reflected beam and produce an oil spill fluorescence spectrum, a programmable device may configure to analyze the oil spill fluorescence spectrum and set an alarm, the programmable device may include one or more processors, at least a memory, a computing program, and at least one connection port, a micro-controller that the micro-controller may configure to control a performance of the light emitting module and digitize the fluorescence spectrum, and a house that the house may configure to hold the light emitting module, the optical receiving device, the detector module, and the micro-controller such that the house may comprise at least an optical window that may configure to provide a path for emission an emitted beam to the surface of water and receiving the reflected beam.

In another yet general aspect, the present disclosure is directed to an exemplary laser remote detection apparatus to detect a non-oil water pollutant. The exemplary laser remote detection apparatus may comprise a light emitting module that the light emitting module may configure to sense a surface of water and induce a fluorescent property of the non-oil water pollutant to produce a non-oil pollutant reflected beam and an optical receiving device which may configure to receive the non-oil pollutant reflected beam such that a tilt angle between the optical receiving device and the light emitting module with respect to an optical axis of the optical receiving device is in a range of 2 to 10 degrees that may configure to adjust a distance between the apparatus and the surface of water. Furthermore, the optical receiving device may include a first lens, at least a filter, at least two second lenses, and at least an optical fiber such that the filter may include a high-pass filter which may configure to pass the non-oil pollutant reflected beam. Moreover, the exemplary laser remote detection apparatus may further comprise a detector module which the detector module may configure to detect the non-oil pollutant reflected beam and produce a non-oil pollutant fluorescence spectrum, a programmable device that may configure to analyze the non-oil pollutant fluorescence spectrum and set an alarm which the programmable device may include one or more processors, at least a memory, a computing program, and at least one connection port, a micro-controller that the micro-controller may configure to control a light emitting module performance and digitize the non-oil pollutant fluorescence spectrum, and a house that the house may configure to encompass the light emitting module, the optical receiving device, the detector module, and the micro-controller. In addition, the house of the exemplary remote detection apparatus may comprise at least an optical window that the optical window may configure to provide a path for emission an emitted beam to the surface of water and receiving the non-oil reflected beam.

### BRIEF DESCRIPTION OF DRAWINGS

The drawing figures depict one or more implementations in accordance with the present teachings, by way of example only, not by way of limitation. In the figures, like reference numerals refer to the same or similar elements.

FIG. 1 illustrates a schematic view of a remote detection apparatus to detect a water pollutant, consistent with one or more exemplary embodiments of the present disclosure.

FIG. 2 illustrates a schematic view of an optical receiving device of a remote detection apparatus to receive a reflected beam, consistent with one or more exemplary embodiments of the present disclosure.

FIG. 3 illustrates an isometric view of an optical receiving device of a remote detection apparatus to receive a reflected beam, consistent with one or more exemplary embodiments of the present disclosure.

FIG. 4 illustrates an isometric view of a cone framework of an optical receiving device, consistent with one or more exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well-known methods, procedures, components, and/or circuitry have been described at a relatively high-level, without detail, in order to avoid unnecessarily obscuring aspects of the present teachings. The following detailed description is, therefore, not to be taken in a limiting sense, and the scope of the present disclosure is defined only by the appended claims.

The following detailed description is presented to enable a person skilled in the art to make and use the methods and apparatuses disclosed in exemplary embodiments of the present disclosure. For purposes of explanation, specific nomenclature is set forth provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

In an exemplary embodiment, a remote detection apparatus may be developed to monitor presence of a pollutant in sea water, rivers, pools, channels in an on-line and real-time mode without contacting a surface of water.

In an exemplary embodiment, an exemplary remote detection apparatus to detect a water pollutant may comprise a light emitting module, an optical receiving device, a detector module, a micro-controller, and a programmable device. In this exemplary embodiment, the exemplary laser remote detection apparatus may further comprise a house that the house may configure to encompass the light emitting module, the optical receiving device, the detector module, and the micro-controller and may configure to protect them from harsh environment conditions.

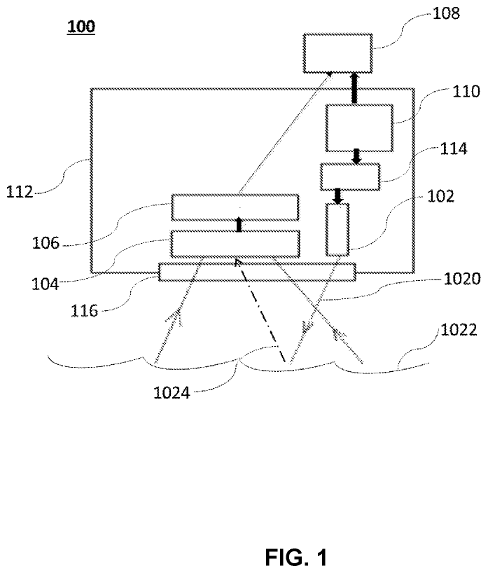

FIG. 1 illustrates an exemplary view of an exemplary remote detection apparatus to detect the water pollutant, consistent with one or more exemplary embodiments of the present disclosure. In an exemplary embodiment, as illustrated in FIG. 1, the exemplary remote detection apparatus 100 may comprise a light emitting module 102, an optical receiving device 104, a detector module 106, a programmable device 108, and a microcontroller 110. In this exemplary embodiment, the exemplary remote detection apparatus 100 may further comprise a house 110 which the light emitting module 102, the optical receiving device 104, the detector module 106, and the micro-controller 110 may be mounted within the house 112. Furthermore, in this exemplary embodiment, the light emitting module 102 may further include a switching key 114 which the light emitting module may be turn on/off by utilizing the switching key 114 in a proper time. In this exemplary embodiment, the switching key 114 may be controlled by utilizing the micro-controller 110 such that the micro-controller 110 may configure to send a receiving command from the programmable device 108 to the switching key 114 for turning off or turning on the light emitting module 102 in the proper time. In this exemplary embodiment, the light emitting module 102 may emit an emitted beam 1020 to a surface of water 1022 that emitted beam 1020 may induce a fluorescent property of the water pollutant that may cause producing a reflected beam 1024 such that the reflected beam1024 may reflect toward the optical receiving device 104. Moreover, in this exemplary embodiment, the house 112 may include an optical window 116 such that the optical window 116 may configure to provide a path for emission of the emitted beam 1020 to the surface of water 1022 and receiving the reflected beam 1024.

In an exemplary embodiment, the house 112 may comprise a first optical window and a second optical window such that both first and second optical window may be mounted on a same side of the house such that the first optical window may configure to provide a first path for emission of the emitted beam from the light emitting module 102 to the surface of water 1022 which the light emitting module 102 may be fixed on the first optical window, the second optical window may configure to provide a second path for receiving the reflected beam from the water pollutant to the optical receiving device 104 such that the optical receiving device 104 may be fixed on the second optical window.

In an exemplary embodiment, the house 112 may be made of a resistant material where may have an anti-corrosion characteristic such that the house 112 may be completely sealed.

In an exemplary embodiment, the remote detection apparatus may further comprise at least three ports where may be mounted on an opposite side of the optical window 116 such that a first port may be configured to connect the detector module 106 to the programmable device 108 by utilizing a cable, a second port may be configured to provide an electrical power for the apparatus 100 by utilizing an electrical cable, and a third port may be configured to connect the micro-controller 110 to the programmable device 108 by utilizing a transferring data cable. In an exemplary embodiment, a wireless port may configure to connect the apparatus 100 to the programable device 108 for transferring data. In an exemplary embodiment, a cable may be, for example, but is not limited to, a network cable. In an exemplary embodiment, the transferring data cable may be, but not limited to, a RS485 cable. In an exemplary embodiment, a communication system that may comprise a wireless system, a Bluetooth system, a GSM system, a Radio-frequency system, and other communication systems that are known by those skilled in the art may be configured to connect the detector module 106 and the micro-controller 110 to the programmable device 108. In an exemplary embodiment, the electrical power of the apparatus 100 may be provided by utilizing a solar cell, a battery, and/or other electrical power systems that are known by those skilled in the art. A consumption power of the apparatus may be in a range of 10 to 50 watts, preferably in a range of 10 to 30 watts, more preferably in a range of 10 to 20 watts.

In another exemplary embodiment, the exemplary remote detection apparatus may further comprise a port where may be mounted on an opposite side of the optical window 116 such that the electrical cable may be connected to the port and provide the electrical power of the apparatus 100. In this exemplary embodiment, a wireless commutation system may be configured to connect the detector module 106 and the micro-controller 110 to the programmable device 108 such that a data transferring may be done through the wireless commutation system between the detector module 106 and the programmable device 108 as well as the micro-controller and the programmable device 108.

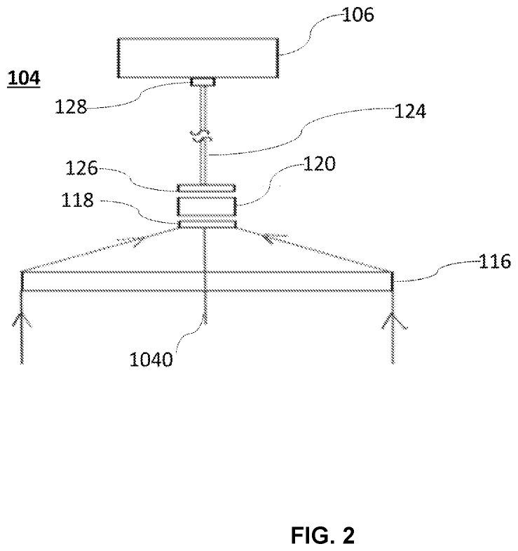

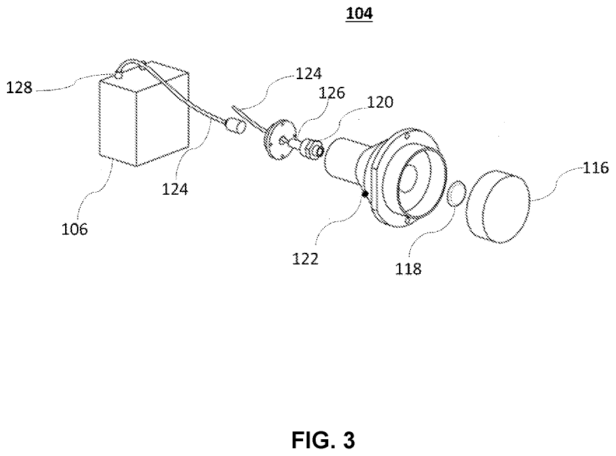

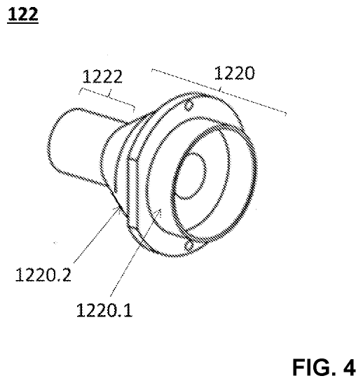

FIG. 2 illustrates an exemplary schematic view of an exemplary optical receiving device 104, consistent with one or more exemplary embodiments of the present disclosure. Furthermore, FIG. 3 illustrates an exemplary isometric view of an exemplary optical receiving device 104 of an exemplary remote detection apparatus to receiving a reflected beam, consistent with one or more exemplary embodiments of the present disclosure. In one or more exemplary embodiment, as illustrated in FIG. 2 and FIG. 3, the exemplary optical receiving device 104 may comprise a lens 116, a filter 118, a collimator 120, and a cone framework 122 (as illustrated in FIG. 4) such that the cone framework 122 may also comprise a first portion 1220 and a second portion 1222 where the first portion 1220 may have a conical shape that may comprise a first frame 1220.1 and a second cone frame 1220.2 such that the lens 116 may be mounted in the first frame 1220.1 of the first portion of cone framework and the second portion 1222 may be connected to the second cone frame 1220.2 of the first portion of framework. In this exemplary embodiment, the lens 116 may configure to collect the reflected beam and a plurality of environmental beams and guide the both reflected and environmental beams to the filter 118 such that the filter may just pass a determined wavelength reflected beam so that the determined wavelength reflected beam may be arranged in a parallel direction in respect with an optical axis 1040 of the optical receiving device through passing the collimator 120 and then an optical fiber 124 that may be connected to the collimator 120 may configure to transfer the parallel beam to the detector module 106. In this exemplary embodiment, two fixed connector 126,128 may configure to connect the collimator to the detector module 106 utilizing the optical fiber 124 such that a first fixed 126 connector may be fixed at an end of a second portion of the cone framework and a second fixed connector 128 may be fixed upon the detector module 106.

In an exemplary embodiment, the optical receiving device 104 may comprise a first lens, at least a filter, at least two second lenses comprising a first collimating lens and a second collimating lens that may configure to arrange the reflected beam to an aligned reflected beam such that the first collimating lens may be located at a confocal length of the first lens and the second collimating lens may be located at a focal length of the first collimating lens, and at least an optical fiber that a framework may configure to encompass the first lens, the filter, and the second lenses such that the first lens may be mounted in a first frame of the framework and the second lenses may be fixed at a second frame of framework such that the first frame may be bigger than the second frame of the framework and the optical fiber 116 may configure to connect the second collimating lens of the second lenses to the detector module 106. In an exemplary embodiment, two optical fibers may be configured to connect the second collimating lens to the detector module 106 such that a first fiber may configure to connect the second collimating lens to a fixed connector at an end of the second frame of the framework and then a second fiber may configure to connect the fixed connector to the detector module 106. In an exemplary embodiment, the focal length may be in a range of, for example, but not limited, 3-12 cm, preferably 3-10 cm. in an exemplary embodiment, the focal length of the first collimating lens may be in a range of, for example, 1-5 cm, preferably 1-3 cm.

In an exemplary embodiment, the remote detection apparatus 100 may further comprise a light spreader such that the light spreader may be mounted in front of the optical emitting module 102 that may configure to spread the emitted light across a wide area of the surface water.

In an exemplary embodiment, an intensity of the emitted beam may be regulated by utilizing the micro-controller.

In an exemplary embodiment, a tilt angle between the light emitting module 102 and the optical receiving device 104 may configure to adjust a distance between the light emitting module 102 and the surface of water 1022. Furthermore, the tilt angle may configure to collect the reflected beam along the optical axis 1040 of the optical receiving device 104. In an exemplary embodiment, the tilt angle may be up to 10 degrees with respect to the optical axis 1040 of the optical receiving device. In an exemplary embodiment, the light emitting module 102 may have the tilt angle in a range of 0 to 10 degrees, preferably 2 to 10 degrees, more preferably up to 2 degree.

In an exemplary embodiment, a distance of 10 meters may be provided with the tilt angle of 4 degrees between the light emitting module 102 and the optical receiving device 104.

In an exemplary embodiment, the light emitting module 102 may be mounted on the cone framework 122 of the optical receiving device 104 in a perpendicular direction with respect to the optical axis 1040 of the optical receiving device 104 such that a reflector mirror may configure to direct the emitted beam along the optical axis of the optical receiving device 1040 to touch the surface of water 1022 and pass the reflected beam to the first collimating lens of the two second lenses 120 such that the reflector mirror may be located within the cone framework 122 of the optical receiving device 104 in front of the light emitting module 102 which the mirror may have an angle in a range of 40 to 50 degree, preferably 45 degree with respect to the optical axis 1040 of the optical receiving device and an optical axis (not illustrated) of the light emitting module. In this exemplary embodiment, the emitted beam that may emit from the light emitting device 102 may hit the mirror then the emitted light 1020 may reflect in a coaxial direction along the optical axis 1040 of the optical receiving device 104 to touch the surface of water 1022.

In an exemplary embodiment, the filter 118 of the optical receiving device may include a specific filter that may just pass a certain wavelength of the fluorescent light such that eliminate other wavelengths of the light to detect a certain water pollutant which may have a fluorescent property. In an exemplary embodiment, a band-pass filter may configure to pass a reflected beam in accordance with an oil pollutant. In other exemplary embodiment, a high-pass filter may configure to pass a reflected beam in accordance with a non-oil water pollutant and eliminate the laser and environmental beam. In an exemplary embodiment, a non-oil water pollutant may be an algae genus, a phytoplankton genus, a microbial pollution in water, or a mixture of two or more thereof.

In an exemplary embodiment, the optical fiber 124 may be a multimode fiber optic. In this exemplary embodiment, a diameter of the multimode fiber optic may be in a range of, for example, 100 to 1200 μm, preferably 300 to 1000 μm. In an exemplary embodiment, the optical fiber may be a single fiber optic.

In an exemplary embodiment, the light emitting module 102 may be, for example, but not limited to, a pulsed diode laser such that the pulsed diode laser may emit a beam with a wavelength of 350-450 nm and a nominal power of 0.05-50 watts that can be turned on and off in a square waveform that can be adjustable with a length of, for example, 1 second to 10 minutes. Also, in an exemplary embodiment, the light emitting module 102 may be modulated by a square waveform and may be adjustable by a modulation rate control circuit. In this exemplary embodiment, the modulation rate control circuit may configure to synchronize the light emitting module 102 with the detector module 106, and a resulting signal is processed. Therefore, a darkness signal, a darkness current, a noise amplifier, and a background noise can be distinguished.

In an exemplary embodiment, a detector module may comprise an inlet slit in a range of 10 to 25 μm, a first mirror, a second mirror, a metal grating plate, a linear array detector, and a linear array processing circuit. In an exemplary embodiment, an arrangement of the detector module may be in a range of 200-800 nm that may be suitable for hydrocarbon pollutants and organic materials in water. In an exemplary embodiment, the mirrors may be parabolic off-axis mirrors. in an exemplary embodiment, the inlet slit may be mounted in a focal length of the first mirror.

In an exemplary embodiment, an exemplary remote detection apparatus 100 to detect an oil spill may comprise the light emitting module 102 configured to sense the surface of water and induce the fluorescent property of the oil spill to produce an oil spill reflected beam, an optical receiving device 104 that may comprise a plano-convex lens, a band-pass filter, at least two bi-convex lenses, and at least the optical fiber such that the band-pass filter may configure to pass the oil spill reflected beam. In this exemplary embodiment, the exemplary apparatus 100 may further comprise the detector module 106 that may configure to detect the oil spill reflected beam and produce a characteristic spectrum according to the oil spill reflected beam. Moreover, in this exemplary embodiment, the laser remote detection apparatus 100 may further comprise a programmable device 108 comprising one or more processors, at least a memory, a computing program, and at least a connection port that may configure to analyze the characteristic spectrum and set an alarm, the micro-controller 110 that may configure to control a performance of the light emitting module and digitize the characteristic spectrum of the oil spill, and the house 112 with the optical window 116 that may configure to protect the light emitting module 102, the optical receiving device 104, the detector module 106, and the micro-controller 110 from the environmental harsh conditions.

In an exemplary embodiment, the light emitting module 102 that may have the tilt angle in the range of 1-10 degrees with respect to the optical axis 1040 of the optical receiving device may be a laser that may have a capability of producing the emitted beam with a wavelength in a range of 370 nm to 470 nm.

In an exemplary embodiment, the light emitting module 102 may be a pulsed diode laser such that the emitted beam may have the wavelength in the range of 370 nm to 470 nm. In an exemplary embodiment, the light emitting module 102 may be mounted within the cone framework 122 of the optical receiving device with a 90-degree angle with respect to the optical axis 1040 of the optical receiving device. In this exemplary embodiment, a reflector mirror may configure to direct the emitted beam 1020 along with the optical axis 1040 of the optical receiving device such that the mirror may have a 45-degree angle with respect to both optical axis of the optical receiving device and the light emitting module.

In an exemplary embodiment, the band-pass filter may be a 480-650 nm band pass filter such that the band pass may pass the reflected beam from the oil spill and eliminate other beams.

In an exemplary embodiment, the two bi-convex lenses may comprise a first bi-convex lens and a second bi-convex lens such that the second bi-convex lens may be mounted at a convex focal length of the first bi-convex lens. In an exemplary embodiment, the convex focal length may be in a range of 1-3 cm. in an exemplary embodiment, the first convex lens may be mounted in a focal length of the plano-convex lens such that the focal length of the plano-convex may be in a range of 8-13 cm.

In another exemplary embodiment, a remote detection apparatus 100 to detect a non-oil water pollutant may comprise a light emitting module 102, an optical receiving device 104 that may comprise a first lens, a high-pass filter, at least two second lenses, and at least an optical fiber such that the high-pass filter may configure to pass a non-oil water pollutant reflected beam and transfer the reflected beam to the first lens of the second lenses, a detector module 106 that may configure to detect the non-oil water pollutant reflected beam and produce a non-oil spectrum, a programmable device 108 that may configure to analyze the non-oil spectrum and set an alarm. In this exemplary embodiment, the remote detection apparatus 100 may further comprise the micro-controller 110 that may configure to send the command of turning on/off to the switching key 114 for controlling the performance of the light emitting module as well as digitize the non-oil spectrum and the house 112 that may include at least an optical window 116 where the light emitting device 102 and the optical receiving device 104 may be mounted within the house 112 in front of the optical window 116. Furthermore, in this exemplary embodiment, the micro-controller 110 may also configure to send a report of an on or off status of the light emitting module 102 to the programable device 108, adjust the alarm, and remove the environmental effects. Also, the house 112 may configure to encompass the detector module 106 and the micro-controller 110 as well as protect the light emitting device 102, the optical receiving device 104, the detector module 106, and the micro-controller 110 from harsh environmental conditions.

In an exemplary embodiment, the light emitting module may be a laser module. In an exemplary embodiment, the laser module may be a pulsed diode module. In an exemplary embodiment, the pulsed diode module may emit an emitted beam in a range of 350 nm to 460 nm.

In an exemplary embodiment, the distance between the apparatus 100 and the surface of water 1022 may be adjusted by utilizing the tilt angle between the light emitting module 102 and the optical receiving device 104. In an exemplary embodiment, the tilt angle may be in a range of 0 to 10 degrees, more preferable between 2 to 10 degrees.

In an exemplary embodiment, the light emitting module 102 may be mounted on the cone framework 122 in a perpendicular position with respect to the optical axis 1040 of the optical receiving device such that the reflected mirror may configure to direct the emitted light from the light 1020 emitting module 102 along the optical axis 1040 and touch the surface of water 1022. in this exemplary embodiment, the reflector mirror may be positioned in front of the light emitting module 102 with the angle in the range of 40-55 degrees with respect to the optical axis 1040 of the optical receiving device and the optical axis (not illustrated) of the light emitting module.

In an exemplary embodiment, the high-pass filter may be a specified high-pass filter in a range of, for example, 370 nm to 470 nm that may configure to pass the reflected beam that may have a wavelength above 370 nm to 470 nm and eliminate the beam with a wavelength below 350 nm to 450 nm. In an exemplary embodiment, the high-pass band may be a 470 nm high-pass filter that may configure to eliminate a beam with a wavelength of the emitted beam 1020 and other beams with a wavelength lower than the wavelength of the emitted beam 1020.

In an exemplary embodiment, the non-oil water pollutant may be a non-oil pollutant with the fluorescent property in the water. In an exemplary embodiment, the non-oil water pollutant may be, for example, but not limited, an algae genus, a phytoplankton genus, a microbial pollution in water, and/or a mixture of two or more thereof.

These connectors can be SMA connectors. Multimode fiber can be 400-1000 μm optical fiber. In the case of using a band-pass filter, detector module (5) can be an avalanche photodiode detector (APD) for detecting oil spills, and in case of using a band-pass filter or high-pass filter, it can be detector box comprising a 20 μm inlet gap, two mirrors, a metal grating plate, a CCD detector, and an array CCD processing circuit that is actually the arrangement of a spectrophotometer in the 200-800 nm range that is suitable for hydrocarbon pollutants and organic materials in water.

### EXAMPLES

In an Example, detection of a crude oil of southern Iran in sea water was carried out with the teachings of the exemplary embodiment of the present disclosure. In this case, an exemplary remote detection apparatus was mounted in a place near the water. A beam from an exemplary laser module of the exemplary apparatus with a wavelength of 450 nm was emitted to the surface of sea water. Following that, in case of presence of the crude oil in the water, the beam exited the fluorescence property of the crude oil and caused producing a reflected beam. Then, a portion of the reflected beam is collected by utilizing an exemplary optical receiving device of the exemplary apparatus such that at first the reflected beam passed through an exemplary lens of the exemplary optical receiving device and the lens directed the reflected beam toward a 530 nm band-pass filter. The 530 nm band-pass filter just passed a 530 nm reflected beam and eliminated another beam in other wavelengths. The 530 nm passed reflected beam then passed through an exemplary collimator of the exemplary optical receiving device and by utilizing an exemplary optical fiber transferred toward an exemplary detector module. The transferred beam passed through an inlet slit of the exemplary detector module and after reflection from a convex mirror, the transferred beam reflect to a reflective grating plate and then enter to a linear array detector by utilizing a second mirror such that the linear array detector is place in a focal length of the second mirror. following that, the produced signal is processed and amplifies by utilizing an AMR circuit. Afterward, the amplified signal is transferred to a personal computer by utilizing a cable and a computing software configured to process the signal and produce a spectrum. The spectrum then compared to an oil specified spectrum and in a case of similarity an alarm is award of presence of the crude oil in the water.

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

It will be understood that the terms and expressions used herein have the ordinary meaning as is accorded to such terms and expressions with respect to their corresponding respective areas of inquiry and study except where specific meanings have otherwise been set forth herein. Relational terms such as first, second, and third and the like may be used solely to distinguish one entity or action from another without necessarily requiring or implying any actual such relationship or order between such entities or actions. The terms “comprises,” “comprising,” “include,” “including, ” or any other variation thereof, are intended to cover a non-exclusive inclusion, such that a process, method, article, apparatus, or device that comprises a list of elements does not include only those elements but may include other elements not expressly listed or inherent to such process, method, article, apparatus, or device. An element proceeded by “a” or “an” does not, without further constraints, preclude the existence of additional identical elements in the process, method, article, or device that comprises the element. Moreover, “may” and other permissive terms are used herein for describing optional features of various embodiments. These terms likewise describe selectable or configurable features generally, unless the context dictates. otherwise.

1. A laser remote detection apparatus to detect a water pollutant comprising:
A light emitting module, the light emitting module configured to sense a surface of water and induce a fluorescent property of the water pollutant to produce a reflected beam;
An optical receiving device, the optical receiving device configured to receive the reflected beam;
A detector module, the detector module configured to detect the reflected beam and produce a fluorescence spectrum;
A programmable device configured to analyze the fluorescence spectrum and set an alarm, the programmable device including one or more processors, at least a memory, a computing program, and at least one connection port; and
A micro-controller, the micro-controller configured to control a performance of the light emitting module and digitize the fluorescence spectrum.

2. The apparatus according to claim 1, further comprising a house configured to hold the light emitting module, the optical receiving device, the detector module, and the micro-controller wherein the house comprises at least an optical window configured to provide a path for transferring a emitted beam to the surface of water and receiving the reflected beam.

3. The apparatus according to claim 1, wherein the light emitting module and the optical receiving device have a tilt angle with respect to an optical axis of the optical receiving device.

4. The apparatus according to claim 1, wherein the light emitting module and the optical receiving device have an angle with respect to an optical axis of the optical receiving device in a range of 2-10 degrees.

5. The apparatus according to claim 1, wherein the light emitting module and the optical receiving device have a 2° tilt angle with respect to an optical axis of the optical receiving device.

6. The apparatus according to claim 1, wherein the laser is a pulsed diode laser.

7. The apparatus according to claim 1, wherein the laser is a pulsed diode laser configured to emit a beam in a range of 350-450 nm.

8. The apparatus according to claim 1, wherein the optical receiving device comprises a first lens, at least a filter, at least two second lenses, and at least an optical fiber;

9. The apparatus according to claim 8, wherein the filter includes a band-pass filter configured to filter an oil pollutant reflected beam.

10. The apparatus according to claim 8, wherein the filter includes a high-pass filter configured to eliminate the emitted beam and pass a non-oil pollutant reflected beam.

11. The apparatus according to claim 1, wherein the two second lenses configured to arrange the beam along the optical axis of the optical receiving device.

12. A laser remote detection apparatus to detect an oil spill comprising:
A light emitting module, the light emitting module configured to sense a surface of water and induce a fluorescent property of the oil spill to produce a reflected beam;
An optical receiving device configured to receive the reflected beam wherein a tilt angle between the optical receiving device and the light emitting module with respect to an optical axis of the optical receiving device is in a range of 2 to 10 degrees configured to adjust a distance of the apparatus and the oil spill, the optical receiving device including a plano-convex lens, at least one filter, at least two bi-convex lenses, and at least an optical fiber, wherein the filter includes a band-pass filter configured to pass an oil spill reflected beam;
A detector module, the detector module configured to detect the oil spill reflected beam and produce a fluorescence spectrum;
A programmable device configured to analyze the fluorescence spectrum and set an alarm, the programmable device including one or more processors, at least a memory, a computing program, and at least one connection port;
A micro-controller, the micro-controller configured to control a light emitting module performance and digitize the fluorescence spectrum; and
A house configured to mount the light emitting module, the optical receiving device, the detector module, and the micro-controller wherein the house comprises at least an optical window configured to provide a path for transferring an emitted beam to the surface of water and receiving the reflected beam.

13. A laser remote detection apparatus to detect an oil spill comprising:
A light emitting module, the light emitting module configured to sense a surface of water and induce a fluorescent property of the oil pollutant to produce a reflected beam;
An optical receiving device configured to receive the reflected beam wherein a tilt angle between the optical receiving device and the light emitting module with respect to an optical axis of the optical receiving device is in a range of 2 to 10 degrees configured to adjust a distance of the apparatus and the oil pollutant, the optical receiving device including a plano-convex lens, at least one filter, at least two bi-convex lenses, and at least an optical fiber, wherein the filter includes a band-pass filter configured to pass an oil spill reflected beam;
A detector module, the detector module configured to detect the oil spill reflected beam and produce an oil spill spectrum;
A programmable device configured to analyze the f oil spill spectrum and set an alarm, the programmable device including one or more processors, at least a memory, a computing program, and at least one connection port;
A micro-controller, the micro-controller configured to control a light emitting module performance and digitize the oil spill spectrum; and
A house configured to mount the light emitting module, the optical receiving device, the detector module, and the micro-controller wherein the house comprises at least an optical window configured to provide a path for transferring an emitted beam to the surface of water and receiving the reflected beam.

14. A laser remote detection apparatus to detect a non-oil water pollutant comprising:
A light emitting module, the light emitting module configured to sense a surface of water and induce a fluorescent property of the non-oil water pollutant to produce a non-oil pollutant reflected beam;
An optical receiving device configured to receive the non-oil pollutant reflected beam wherein a tilt angle between the optical receiving device and the light emitting module with respect to an optical axis of the optical receiving device is in a range of 2 to 10 degrees configured to adjust a distance between the apparatus and the oil pollutant, the optical receiving device including a first lens, at least one filter, at least two second lenses, and at least an optical fiber, wherein the filter includes a high-pass filter configured to pass a non-oil pollutant reflected beam;
A detector module, the detector module configured to detect the non-oil pollutant reflected beam and produce a non-oil pollutant fluorescence spectrum;
A programmable device configured to analyze the non-oil pollutant fluorescence spectrum and set an alarm, the programmable device including one or more processors, at least a memory, a computing program, and at least one connection port;
A micro-controller, the micro-controller configured to control a performance of the light emitting module and digitize the non-oil fluorescence spectrum; and
A house configured to mount the light emitting module, the optical receiving device, the detector module, and the micro-controller wherein the house comprises at least an optical window configured to provide a path for emission an emitted beam to the surface of water and receiving the non-oil pollutant reflected beam.
